(19) United States
(12) Patent Application Publication
(10) Pub. No.: US 2025/0206711 A1
(43) Pub. Date: Jun. 26, 2025

(54) PROCESS FOR PRODUCING 5-HYDROXYMETHYLFURFURAL USING METAL PHOSPHATES

(71) Applicant: UOP LLC, Des Plaines, IL (US)

(72) Inventors: Steven L. Chmura, Des Plaines, IL (US); Erin M. Broderick, Arlington Heights, IL (US); Gregory B. Kuzmanich, Arlington Heights, IL (US)

(21) Appl. No.: 18/890,832

(22) Filed: Sep. 20, 2024

Related U.S. Application Data
(60) Provisional application No. 63/613,088, filed on Dec. 21, 2023.

(51) Int. Cl. C07D 307/50; B01J 27/18

(52) U.S. Cl. CPC C07D 307/50; B01J 27/1802

(57) ABSTRACT

Processes for synthesizing 5-hydroxymethylfurfural are described. The processes comprise contacting a feed comprising biomass-derived cellulose, or a sugar monomer or oligomer, or a mixture thereof with a catalyst comprising a metal phosphate. The metal phosphate may comprise hafnium phosphate, or zirconium phosphate, or combinations thereof. The catalyst has a ratio of Bronsted acid sites to Lewis acid sites greater than or equal to 0.27 and a total acid density less than or equal to 0.4.

### RELATED APPLICATIONS

This application claims priority to U.S. Provisional Patent Application Ser. No. 63/613,088, filed on Dec. 21, 2023, the entirety of which is incorporated herein by reference.

### BACKGROUND

5-hydroxymethylfurfural (5-HMF) is a major platform chemical generated from lignocellulosic biomass that can be transformed into a versatile building block for a variety of green chemicals and fuels.

Monosaccharides, which are more expensive carbohydrates, are routinely converted to HMF. Cellulose, a more abundant and less expensive carbohydrate, can be transformed to produce HMF as a renewable route to biofuels and fine chemicals.

HMF is commercially produced from lignocellulose found in various herbaceous and woody biomass sources. Lignocellulose is the structural component of plant cell walls and is a complex mix of three main components: cellulose, hemicellulose, and lignin.

Cellulose is a linear chain of several hundred to many thousands of β linked D-glucose units. Hemicellulose is a heteropolymer, present along with cellulose in almost all terrestrial plant cell walls. While cellulose is crystalline, strong, and resistant to hydrolysis, hemicelluloses have random, amorphous structures with little strength. Lignin is a polymeric material that is a cross-linked component of three monolignols: coumaroyl alcohol, coniferyl alcohol, and sinapyl alcohol. It is thus a highly aromatic polymer. The different polymers exhibit different reactivity to thermal, chemical, and biological processing.

The traditional process for producing HMF from biomass involves hot acid digestion to hydrolyze the hemicellulose to release the C₅ sugars followed by acid catalyzed isomerization and dehydration of the C₅ sugars to furfural. However, many side reactions can occur due to the complex structure of lignocellulose and the acidic environment. In addition to isomerization and dehydration of the C₅ sugars, a similar process can isomerize the C₆ sugar component of hemicellulose or perhaps even some C₆ sugars from cellulose to 5-HMF which may undergo further hydrolysis to levulinic and formic acids. Acetic acid is also generated from the acetyl groups on the hemicellulose. Furthermore, both furfural and 5-HMF can also undergo subsequent polymerization to form humins which are highly crosslinked chains of furans and hexose sugars.

However, the yields for commercial processes of producing HMF are only about 50% of theoretical, leading to high production costs.

Therefore, there is a need for improved and less expensive processes for making 5-HMF from biomass.

### BRIEF DESCRIPTION OF THE DRAWINGS

FIG. 1 is an illustration of one embodiment of a process for producing HMF from biomass.

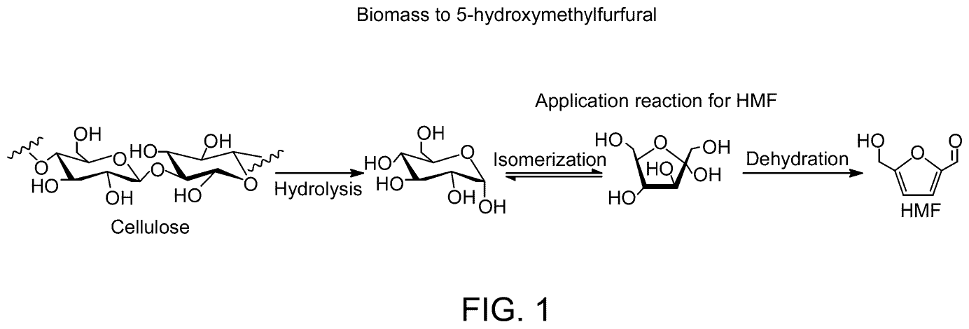

FIG. 2 is a graph showing selectivity as a function of glucose conversion to HMF with zirconium phosphates as the catalyst.

FIG. 3 is a graph showing selectivity as a function of cellulose conversion to HMF with hafnium and zirconium phosphates catalysts.

### DETAILED DESCRIPTION

The present process involves processes for synthesizing 5-hydroxymethylfurfural. In some embodiments, the processes comprise contacting a feed comprising biomass-derived cellulose, or a sugar monomer or oligomer, or combinations thereof with a catalyst comprising a metal phosphate. The catalyst has a ratio of Bronsted acid sites to Lewis acid sites greater than or equal to 0.27 and a total acid density less than or equal to 0.4.

An infrared measurement is performed by first pretreating the sample at 500° C. in flowing helium for 2 hours. The sample is cooled to room temperature to take the spectrum. Pyridine adsorption was performed at 150° C. for one hour followed by discrete desorptions at 150° C., 300° C., and 450° C. with the spectra taken at room temperature following each discrete desorption. The species from wavenumbers of 1568 to 1510 cm⁻¹ are assigned as Bronsted acid species and species from wave numbers of 1473 to 1430 cm⁻¹ are assigned as Lewis acid species. The areas under this specific range is integrated and normalized to area per milligram.

In some embodiments, the feed comprises biomass-derived cellulose. The biomass-derived cellulose may comprise native lignocellulosic material, or pretreated lignocellulosic material, or microcrystalline cellulose, or nanocrystalline cellulose, or combinations thereof. In some embodiments, the feed comprises a mixture of biomass-derived cellulose and one or more sugar monomers and/or oligomers. In some embodiments, the feed comprises one or more sugar monomers and/or oligomers. The sugar oligomers may comprise oligosaccharides having between 3 and 10 sugar residues, or disaccharides, or combinations thereof. Sugar monomers are monosaccharides including, but not limited to, glucose, fructose, xylose, galactose, and the like, and their isomers.

In some embodiments, the metal phosphate comprises hafnium phosphate, or zirconium phosphate, or combinations thereof. In some embodiments, the metal phosphate has a mol ratio of phosphorus to metal in a range of 0.1:1 to 10:1.

The feed may be combined with water and optionally a solvent. When the solvent is present, the mole ratio of solvent to water may be in the range of 0.01:1 to 100:1. Suitable solvents include, but are not limited to, cyclic ethers, alcohols, sulfoxides, ketones, or combinations thereof. Suitable cyclic ethers include, but are not limited to, tetrahydrofuran, methyl tetrahydrofuran, dioxane, dimethylfuran, or combinations thereof. Suitable alcohols include, but are not limited to ethanol, butanol, and the like. Suitable sulfoxides include, but are not limited to, dimethyl sulfoxide. Suitable ketones include, but are not limited to, methyl isobutylketone, gamma valero lactone, or combinations thereof.

The water may include a salt. Typically, the mole ratio of the salt to the water is in the range of 0.001:1 to 0.5:1. Suitable solvents include, but are not limited to, sodium chloride, lithium chloride, potassium chloride, cesium chloride, magnesium chloride, calcium chloride, or combinations thereof.

The concentration of the feed in the water and solvent may be in the range of 0.01 wt % to 20 wt %.

Suitable operating conditions include, but are not limited to, temperatures in the range of 100° C. to 250° C., pressures in the range of 0 MPa to 6.9 MPa, or both. Suitable contact times include a range of 1 sec to 24 hr.

The process may comprise batch processes, continuous processes, or semi-continuous processes.

The process may take place in a single reactor or in multiple reactors (two or more). In some embodiments, the biomass-derived cellulose may be contacted with the metal phosphate catalyst in a first reactor, resulting in the biomass-derived cellulose being converted to 5-hydroxymethylfurfural and sugar monomers and/or oligomers. The sugar monomers and/or oligomers produced in the first reactor may be contacted with a second catalyst in a second reactor resulting in the sugar monomers and/or oligomers being converted to additional 5-hydroxymethylfurfural in the second reactor. In this arrangement, the first and second catalysts may be the same, or they may be different. Any suitable catalyst can be used for the second catalyst. Suitable catalysts include, but are not limited to, metal phosphates, aluminosilicate zeolites, silica aluminophosphate zeolites, zirconium sulfates, homogenous acids, metal oxides, or combinations thereof.

The catalyst comprising the metal phosphate and the second catalyst may optionally include a binder such as silica or alumina, as is well known in the art.

In some embodiments, the conversion of the biomass-derived cellulose and/or sugar monomers and/or oligomers is greater than or equal to 75%, or 80%, or 85%, or 90%. In some embodiments, the yield of 5-hydroxymethylfurfural is greater than or equal to 20%, or 25%, or 30%, or 35%, or 40%. In some embodiments, the conversion of the biomass-derived cellulose and/or sugar monomers and/or oligomers is greater than or equal to 75%, and the yield of 5-hydroxymethylfurfural is greater than or equal to 20%. In some embodiments, the conversion of the biomass-derived cellulose and/or sugar monomers and/or oligomers is greater than or equal to 90%, and the yield of 5-hydroxymethylfurfural is greater than or equal to 30%. In some embodiments, the conversion of the biomass-derived cellulose and/or sugar monomers and/oligomers is greater than or equal to 90%, and the yield of 5-hydroxymethylfurfural is greater than or equal to 35%. In some embodiments, the conversion of the biomass-derived cellulose and/or sugar monomers and/or oligomers is greater than or equal to 90%, and the yield of 5-hydroxymethylfurfural is greater than or equal to 40%.

Another aspect of the invention is a process for synthesizing 5-hydroxymethylfurfural. In one embodiment, the process comprises contacting a feed comprising biomass-derived cellulose, or a sugar monomer or oligomer, or a mixture thereof with a catalyst in the presence of water and a solvent, wherein the catalyst comprises a metal phosphate wherein the metal phosphate comprises hafnium phosphate, or zirconium phosphate, or combinations thereof, wherein the catalyst has a ratio of Bronsted acid sites to Lewis acid sites greater than or equal to 0.27 and a total acid density less than or equal to 0.4; and wherein the feed is contacted with the catalyst at a temperature in a range of 100° C. to 250° C., or at a pressure in a range of 0 MPa to 6.9 MPa, or for a time in a range of 1 sec to 24 hr, or combinations thereof.

In some embodiments, the process include one reactor, or two (or more) reactors. The biomass-derived cellulose feed can be contacting with the first catalyst in the first reactor where the biomass-derived cellulose is converted to 5-hydroxymethylfurfural and sugar monomers and/or oligomers. The sugar monomers and/or oligomers produced in the first reactor can be contacted with a second catalyst in the second reactor where the sugar monomers and/or oligomers are converted to additional 5-hydroxymethylfurfural. Sugar monomers contacted with the catalyst would be converted to 5-hydroxymethylfurfural, and any unconverted sugar monomers would be recycled.

The starting materials, catalysts, and reaction conditions are discussed above.

### EXAMPLES

### Example 1

Catalyst synthesis: Two aqueous solutions were prepared. A potassium dihydrogen phosphate (KH₂PO₄) solution was prepared by adding KH₂PO₄ (2.72 g) to 50 mL water and allowing it to dissolve with stirring. A metal chloride solution was prepared by adding metal chloride (3.20 g HfCl₄ or 2.33 g ZrCl₄) to 50 ml of water and allowing it to dissolve with stirring. The KH₂PO₄ solution was added dropwise to the aqueous metal chloride solution with stirring. The mixture was stirred for two hours at room temperature and then aged in a Teflon lined autoclave at 120° C. for six hours. The catalyst was recovered through filtration and thoroughly washed with water and ethanol. The catalyst was dried at 80° C. overnight in air. All catalyst samples were calcined in an air atmosphere at 550° C. for four hours.

Metal phosphate testing: For the conversion of cellulose or glucose, experimentation was carried out in a THF/H₂O solvent system (20 mL THF/H₂O, 4:1 volume ratio), a mixture of microcrystalline cellulose or glucose (0.4 g), catalyst (30 wt % by mass of cellulose), and NaCl (20 wt % by mass of water) were loaded into an autoclave. The autoclave was purged with N₂ three times to remove oxygen. The reaction mixture was heated to 190° C. for four hours for cellulose, and 175° C. for 2.5 hours for glucose. The product was filtered and was allowed to separate into two phases.

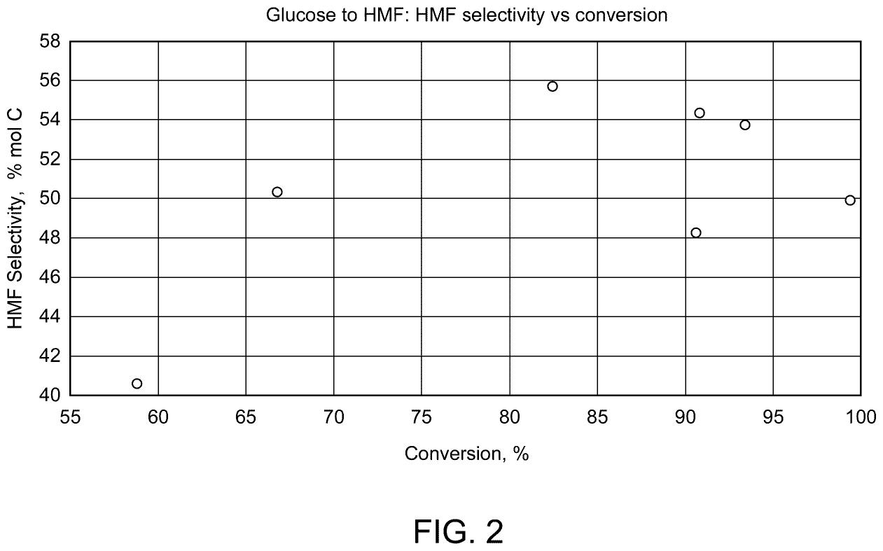

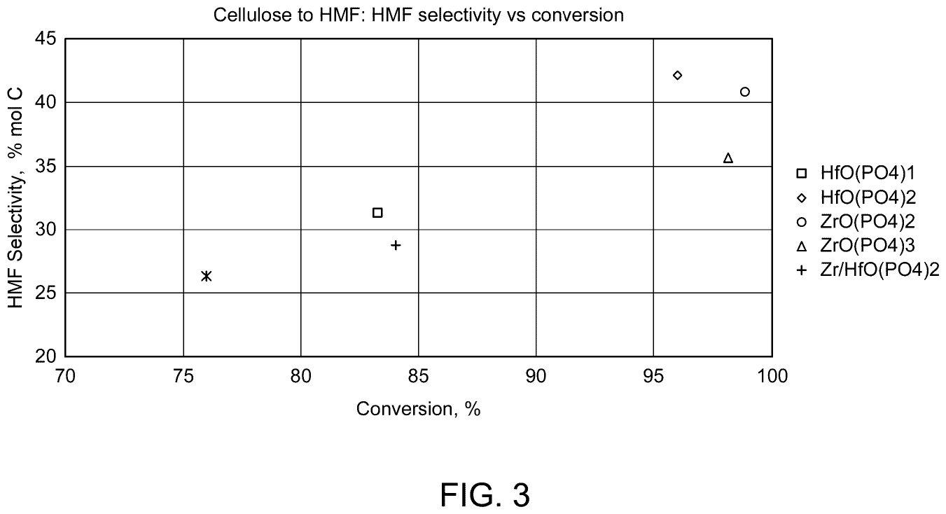

The zirconium phosphate catalyst converted glucose with up to 93% conversion and 50% HMF yield (FIG. 2) depending upon the experimental conditions. The metal phosphate catalysts converted cellulose to HMF (FIG. 3) in varying yields depending upon variables of the material, such as metal:P ratio, Bronsted acid:Lewis acid ratio, and the number of acid sites. The best HMF yields from cellulose were obtained with a catalyst that has a ratio of Bronsted acid sites to Lewis acid sites greater than or equal to 0.27 and a total acid density less than or equal to 0.4 (Table 1).

TABLE 1
Catalyst Properties

| Catalyst | HfO(PO₄)₁ | HfO(PO₄)₂ | ZrO(PO₄)₁ | ZrO(PO₄)₂ |
|---|---|---|---|---|
| BET surface area | 172 | 176 | 242 | 141 |
| Pore volume, cm³/g | 0.385 | 0.387 | 0.576 | 0.528 |
| PyB/PyL Acidity, | 0.1 | 0.436 | 0.133 | 0.424 |
| n/n | | | | |
| Total acid density, | 0.285 | 0.254 | 0.349 | 0.126 |
| mmol/g | | | | |
| Strong acid density, | 0.285 | 0 | 0.349 | 0 |
| mmol/g | | | | |
| Cellulose | 83 | 96 | 76 | 99 |
| conversion | | | | |
| HMF yield | 26 | 40 | 20 | 40 |
| Glucose conversion | | | | 93 |
| HMF yield | | | | 50 |

### Example 2

Batch autoclave experiments were performed in a 75 mL autoclave with a salt bath. In a typical experiment, the sugar solution (0.4 g C₆ sugar in 4 g water) and sodium chloride (0.8 g) were mixed. The water solution was loaded into the autoclave followed by tetrahydrofuran (14 g THF). The catalyst (40-60 mesh of ZrO(PO₄)₂, 0.094 g) was added to the autoclave, which was sealed and placed into the salt bath. After stirring for the set amount of time (30 min in 275° C. salt bath with internal temperature maximum at 212° C.), the autoclave was removed from the salt bath and placed into an ice bath. The solution was centrifuged to separate the liquid from the solid phases. The organic and aqueous phases were also decanted via a pipette. Samples were submitted for both phases for LC analysis. The HMF yield was greater than 60%.

### SPECIFIC EMBODIMENTS

While the following is described in conjunction with specific embodiments, it will be understood that this description is intended to illustrate and not limit the scope of the preceding description and the appended claims.

A first embodiment of the invention is a process for synthesizing 5-hydroxymethylfurfural comprising contacting a feed comprising biomass-derived cellulose, or a sugar monomer or oligomer, or a mixture thereof with a catalyst comprising a metal phosphate, wherein the catalyst has a ratio of Bronsted acid sites to Lewis acid sites greater than or equal to 0.27 and a total acid density less than or equal to 0.4. An embodiment of the invention is one, any or all of prior embodiments in this paragraph up through the first embodiment in this paragraph wherein the metal phosphate has a mol ratio of phosphorus to metal in a range of 0.11 to 101. An embodiment of the invention is one, any or all of prior embodiments in this paragraph up through the first embodiment in this paragraph wherein the metal phosphate comprises hafnium phosphate, or zirconium phosphate, or combinations thereof. An embodiment of the invention is one, any or all of prior embodiments in this paragraph up through the first embodiment in this paragraph wherein contacting the feed with the catalyst comprises contacting the feed with the catalyst in the presence of water and optionally a solvent. An embodiment of the invention is one, any or all of prior embodiments in this paragraph up through the first embodiment in this paragraph wherein the solvent is present and wherein a mole ratio of the solvent to the water is in a range of 0.011 to 1001. An embodiment of the invention is one, any or all of prior embodiments in this paragraph up through the first embodiment in this paragraph wherein the solvent is present and wherein the solvent comprises a cyclic ether, an alcohol, a sulfoxide, a ketone, or combinations thereof. An embodiment of the invention is one, any or all of prior embodiments in this paragraph up through the first embodiment in this paragraph wherein the water contains a salt and wherein a mole ratio of the salt to the water is in a range of 0.0011 to 0.51. An embodiment of the invention is one, any or all of prior embodiments in this paragraph up through the first embodiment in this paragraph wherein the salt comprises sodium chloride, lithium chloride, potassium chloride, cesium chloride, magnesium chloride, calcium chloride, or combinations thereof. An embodiment of the invention is one, any or all of prior embodiments in this paragraph up through the first embodiment in this paragraph wherein contacting the feed with the catalyst takes place at a temperature in a range of 100° C. to 250° C., or at a pressure in a range of 0 MPa to 6.9 MPa, or both. An embodiment of the invention is one, any or all of prior embodiments in this paragraph up through the first embodiment in this paragraph wherein contacting the feed with the catalyst comprises contacting the feed with the catalyst for a time in a range of 1 sec to 24 hr, An embodiment of the invention is one, any or all of prior embodiments in this paragraph up through the first embodiment in this paragraph wherein contacting the feed with the catalyst takes place in a batch process, or a continuous process, or a semi-continuous process. An embodiment of the invention is one, any or all of prior embodiments in this paragraph up through the first embodiment in this paragraph wherein contacting the feed with the catalyst comprising the metal phosphate comprises contacting the biomass-derived cellulose with the catalyst comprising the metal phosphate in a first reactor and wherein the biomass-derived cellulose is converted to 5-hydroxymethylfurfural and sugar monomers and/or oligomers in the first reactor; and contacting the sugar monomers and/or oligomers produced in the first reactor with a second catalyst in a second reactor and wherein the sugar monomers and/or oligomers are converted to additional 5-hydroxymethylfurfural in the second reactor. An embodiment of the invention is one, any or all of prior embodiments in this paragraph up through the first embodiment in this paragraph wherein the catalyst comprising the metal phosphate and the second catalyst are different. An embodiment of the invention is one, any or all of prior embodiments in this paragraph up through the first embodiment in this paragraph wherein a concentration of the feed in water and a solvent is in a range of 0.01 wt % to 20 wt %. An embodiment of the invention is one, any or all of prior embodiments in this paragraph up through the first embodiment in this paragraph wherein the biomass-derived cellulose comprises native lignocellulosic material, or pretreated lignocellulosic material, or microcrystalline cellulose, or nanocrystalline cellulose, or combinations thereof or wherein the sugar oligomers comprises oligosaccharides between 3 and 10 sugar residues, or disaccharides, or combinations thereof; or both.

A second embodiment of the invention is a process for synthesizing 5-hydroxymethylfurfural comprising contacting a feed comprising biomass-derived cellulose, or a sugar monomer or oligomer, or a mixture thereof with a catalyst in the presence of water and a solvent, wherein the catalyst comprises a metal phosphate wherein the metal phosphate comprises hafnium phosphate, or zirconium phosphate, or combinations thereof, wherein the catalyst has a ratio of Bronsted acid sites to Lewis acid sites greater than or equal to 0.27 and a total acid density less than or equal to 0.4; and wherein the feed is contacted with the catalyst at a temperature in a range of 100° C. to 250° C., or at a pressure in a range of 0 MPa to 6.9 MPa, or for a time in a range of 1 sec to 24 hr, or combinations thereof. An embodiment of the invention is one, any or all of prior embodiments in this paragraph up through the second embodiment in this paragraph wherein contacting the feed with the catalyst comprising the metal phosphate comprises contacting the biomass-derived cellulose with the catalyst comprising the metal phosphate in a first reactor and wherein the biomass-derived cellulose is converted to 5-hydroxymethylfurfural and sugar monomers and/or oligomers in the first reactor; and contacting the sugar monomers and/or oligomers produced in the first reactor with a second catalyst in a second reactor and wherein the sugar monomers and/or oligomers are converted to additional 5-hydroxymethylfurfural in the second reactor. An embodiment of the invention is one, any or all of prior embodiments in this paragraph up through the second embodiment in this paragraph wherein the catalyst comprising the metal phosphate and the second catalyst are different. An embodiment of the invention is one, any or all of prior embodiments in this paragraph up through the second embodiment in this paragraph wherein the metal phosphate has a mol ratio of phosphorus to metal in a range of 0.11 to 101; or wherein a mole ratio of the solvent to the water is in a range of 0.011 to 1001; or wherein the water contains a salt and wherein a mole ratio of the salt to the water is in a range of 0.0011 to 0.51; or wherein a concentration of the feed in water and a solvent is in a range of 0.01 wt % to 20 wt %; or combinations thereof. An embodiment of the invention is one, any or all of prior embodiments in this paragraph up through the second embodiment in this paragraph wherein the solvent is present and wherein the solvent comprises a cyclic ether, an alcohol, a sulfoxide, a ketone, or combinations thereof, or combinations thereof.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A process for synthesizing 5-hydroxymethylfurfural comprising:
contacting a feed comprising biomass-derived cellulose, or a sugar monomer or oligomer, or a mixture thereof with a catalyst comprising a metal phosphate, wherein the catalyst has a ratio of Bronsted acid sites to Lewis acid sites greater than or equal to 0.27 and a total acid density less than or equal to 0.4.

2. The process of claim 1 wherein the metal phosphate has a mol ratio of phosphorus to metal in a range of 0.1:1 to 10:1.

3. The process of claim 1 wherein the metal phosphate comprises hafnium phosphate, or zirconium phosphate, or combinations thereof.

4. The process of claim 1 wherein contacting the feed with the catalyst comprises contacting the feed with the catalyst in the presence of water and optionally a solvent.

5. The process of claim 4 wherein the solvent is present and wherein a mole ratio of the solvent to the water is in a range of 0.01:1 to 100:1.

6. The process of claim 4 wherein the solvent is present and wherein the solvent comprises a cyclic ether, an alcohol, a sulfoxide, a ketone, or combinations thereof.

7. The process of claim 4 wherein the water contains a salt and wherein a mole ratio of the salt to the water is in a range of 0.001:1 to 0.5:1.

8. The process of claim 7 wherein the salt comprises sodium chloride, lithium chloride, potassium chloride, cesium chloride, magnesium chloride, calcium chloride, or combinations thereof.

9. The process of claim 1 wherein contacting the feed with the catalyst takes place at a temperature in a range of 100° C. to 250° C., or at a pressure in a range of 0 MPa to 6.9 MPa, or both.

10. The process of claim 1 wherein contacting the feed with the catalyst comprises contacting the feed with the catalyst for a time in a range of 1 sec to 24 hr.

11. The process of claim 1 wherein contacting the feed with the catalyst takes place in a batch process, or a continuous process, or a semi-continuous process.

12. The process of claim 1 wherein contacting the feed with the catalyst comprising the metal phosphate comprises:
contacting the biomass-derived cellulose with the catalyst comprising the metal phosphate in a first reactor and wherein the biomass-derived cellulose is converted to 5-hydroxymethylfurfural and sugar monomers or oligomers or both in the first reactor; and
contacting the sugar monomers or oligomers or both produced in the first reactor with a second catalyst in a second reactor and wherein the sugar monomers or oligomers or both are converted to additional 5-hydroxymethylfurfural in the second reactor.

13. The process of claim 12 wherein the catalyst comprising the metal phosphate and the second catalyst are different.

14. The process of claim 1 wherein a concentration of the feed in water and a solvent is in a range of 0.01 wt % to 20 wt %.

15. The process of claim 1:
wherein the biomass-derived cellulose comprises native lignocellulosic material, or pretreated lignocellulosic material, or microcrystalline cellulose, or nanocrystalline cellulose, or combinations thereof: or
wherein the sugar oligomers comprises oligosaccharides between 3 and 10 sugar residues, or disaccharides, or combinations thereof;
or both.

16. A process for synthesizing 5-hydroxymethylfurfural comprising:
contacting a feed comprising biomass-derived cellulose, or a sugar monomer or oligomer, or a mixture thereof with a catalyst in the presence of water and a solvent, wherein the catalyst comprises a metal phosphate wherein the metal phosphate comprises hafnium phosphate, or zirconium phosphate, or combinations thereof, wherein the catalyst has a ratio of Bronsted acid sites to Lewis acid sites greater than or equal to 0.27 and a total acid density less than or equal to 0.4; and wherein the feed is contacted with the catalyst at a temperature in a range of 100° C. to 250° C., or at a pressure in a range of 0 MPa to 6.9 MPa, or for a time in a range of 1 sec to 24 hr, or combinations thereof.

17. The process of claim 16 wherein contacting the feed with the catalyst comprising the metal phosphate comprises:
contacting the biomass-derived cellulose with the catalyst comprising the metal phosphate in a first reactor and wherein the biomass-derived cellulose is converted to 5-hydroxymethylfurfural and sugar monomers or oligomers or both in the first reactor; and
contacting the sugar monomers to oligomers or both produced in the first reactor with a second catalyst in a second reactor and wherein the sugar monomers or oligomers or both are converted to additional 5-hydroxymethylfurfural in the second reactor.

18. The process of claim 17 wherein the catalyst comprising the metal phosphate and the second catalyst are different.

19. The process of claim 16:
wherein the metal phosphate has a mol ratio of phosphorus to metal in a range of 0.1:1 to 10:1; or
wherein a mole ratio of the solvent to the water is in a range of 0.01:1 to 100:1; or
wherein the water contains a salt and wherein a mole ratio of the salt to the water is in a range of 0.001:1 to 0.5:1; or
wherein a concentration of the feed in water and a solvent is in a range of 0.01 wt % to 20 wt %;
or combinations thereof.

20. The process of claim 16 wherein the solvent is present and wherein the solvent comprises a cyclic ether, an alcohol, a sulfoxide, a ketone, or combinations thereof, or combinations thereof.
